(19) United States
(12) Patent Application Publication
(10) Pub. No.: US 2014/0088504 A1
(43) Pub. Date: Mar. 27, 2014

(54) HANDHELD MEDICAMENT DELIVERY DEVICE WITH DOSE BUTTON

(71) Applicant: SANOFI-AVENTIS DEUTSCHLAND GMBH, Frankfurt (DE)

(72) Inventor: Stuart King, West Midlands (GB)

(21) Appl. No.: 14/119,212

(22) PCT Filed: May 24, 2012
(86) PCT No.: PCT/EP2012/059754

§ 371 Date: Nov. 21, 2013

(30) Foreign Application Priority Data
May 25, 2011 (EP) 11167535

(51) Int. Cl. A61M 5/24; A61M 5/31; A61M 5/315

(52) U.S. Cl. CPC A61M 5/24; A61M 5/3159; A61M 5/3137
USPC 604/131

(57) ABSTRACT

The invention resides in a hand-held medicament delivery device having a housing in which is mounted, a switch, an electro-mechanical drug delivery mechanism activatable by the switch and a dose button associated with the switch. When the dose button is pressed by a user, the switch is activated. Furthermore, the dose button is hinged along one edge thereof. The dose button may be hinged about a support element of the housing or integrally formed with at least a part of the housing, a line of weakness such as a thin walled region being provided between the housing and the dose button to serve as the hinge.

### CROSS REFERENCE TO RELATED APPLICATIONS

The present application is a U.S. National Phase Application pursuant to 35 U.S.C. §371 of International Application No. PCT/EP2012/059754 filed May 24, 2012, which claims priority to European Patent Application No. 11167535.1 filed May 25, 2011. The entire disclosure contents of these applications are herewith incorporated by reference into the present application.

### TECHNICAL FIELD

The present invention relates to an improved dose button, forming part of a handheld medicament delivery device.

### BACKGROUND

Certain medical conditions require patients to self-administer medicament(s) over a long period of time, perhaps years. Where possible such medicaments will be formulated for oral delivery which helps with patient compliance. Due to the nature of the medicament (e.g. insulin) oral delivery is not always possible and other administration routes are necessary. Self administration by injection for chronic conditions such as diabetes is therefore relatively common.

Over recent years there has been significant development in the area of injectors. In particular electro-mechanical injectors are now available. Such devices are generally battery powered and designed for multiple uses. The devices generally comprise a housing having an electro-mechanical drug delivery mechanism, such as a motor-driven piston which acts on a cartridge containing the medicament to be delivered through a needle attached to the device. Needleless injectors such as jet injectors are also known. The devices commonly have a graphical display for displaying such information as device status (e.g. ready for injection, cartridge empty, error status, dosing history etc.) a user interface usually in the form of a number of buttons) for entering a required dose, initiating dosing and/or priming and powering up/down the device and a microprocessor for controlling the drug delivery mechanism according to a user defined dose, monitoring error conditions, writing dose histories to memory etc.

These sophisticated electro-mechanical devices are required to be sufficiently robust to survive such hazards as moisture and dust ingress which are likely to occur in a domestic environment.

Depending on the injection site, one hand operation may be required and/or the device may only be partially or not at all visible. Thus the dose button (sometimes referred to as an injection button) must be reliable and provide sufficient tactile feedback to the user that a dose has been initiated. Moreover, users may lack manual dexterity, have visual impairment and/or suffer weakness in the hands so ease of operation is important.

The present invention is conceived with the above problems in mind.

### SUMMARY

According to the present invention there is provided a hand-held medicament delivery device having a housing in which is mounted,

a switch,

an electro-mechanical drug delivery mechanism activatable by the switch,

a dose button associated with the switch such that pressing of the dose button by a user activates the switch,

wherein the dose button is hinged along one edge thereof.

It will be understood that in use, movement of the dose button is constrained to pivoting about the hinged edge.

In certain embodiments the device housing will have a front face incorporating a graphical display and an end face incorporating the dose button contiguous with and substantially perpendicular to the front face, in which case the dose button is conveniently hinged substantially on the interface between the front and end faces. It will be understood that hinging the button in such a way allows convenient operation of the dose button with a finger or thumb of either hand without obscuring the display.

In an alternative embodiment, the dose button is hinged on the interface between the end face, on a side face or on a back face of the device.

In certain embodiments the device is an injection device, such as a needle injector.

In certain embodiments a hinge is formed by mounting the dose button on a rod or posts or other support element of the device housing. Alternatively, the dose button can be integrally formed with at least a part of the housing (for example a part of the front face), a line of weakness such as a thin walled region being provided between the housing and the dose button to serve as a hinge.

In an example embodiment, the switch is a dome switch, although in some embodiments the switch may be a microswitch or a plurality of such switches.

In certain embodiments, a gasket is provided between the switch and the dose button. Such gasket improves the sealing of the device against moisture and dust/dirt. Conveniently the gasket is in the form of a flexible membrane made from for example silicone rubber. Alternatives include other flexible materials such as a TPE (thermoplastic elastomer) or TPU (thermoplastic polyurethanes)

In known medicament delivery devices (e.g. EasyPod™—manufactured and marketed by Merck Serono), the dose button is a standard “free-floating” button, i.e. when pressed the button travel is generally perpendicular to the plane of the button such a dose button has a number of disadvantages:
When pressed, the gap between the housing and the button edge corresponds to the full travel distance of the button around the entire edge of the button. Not only does this expose a large area for the potential of ingress of moisture or dust, it leads to the possibility of the button jamming.
Since the button can tilt in any direction, there is significant variance in the amount of force required to activate the underlying switch depending on exactly where the user presses the button.
The button must be relatively small to avoid “dead spots” where pressing of the button fails to activate the switch. The alternative is to provide a more complicated arrangement with perhaps multiple switches.

In contrast, the use of a hinged dose button offers the following advantages:
The gap corresponding to the full travel of the button is only present along a single edge of the button. There is no increase in gap along the hinged edge and along the edges contiguous with the hinged edge, the gap is on average only half the distance corresponding to the full travel of the button. As a result the potential for ingress of moisture and dust is reduced as is the potential for jamming.
The hinged button offers a more consistent and reliable action. There are far fewer or no dead spots.

As a result of the above advantages, it is possible to use a relatively large dose button which assists with usability of the device. For example, the dose button may occupy at least 30%, at least 40%, at least 50%, at least 60%, at least 70% or at least 80% of the face of the device in which it is located.

In certain embodiments the dose button is associated with a single switch.

In certain embodiments the device also includes a programmable microprocessor having memory and input/output functions.

In certain embodiments the device also includes one or more additional buttons (which may be of the hinged or free floating variety) serving as a user interface for programming the device.

In the case of a needle injection device, the housing will also include a mounting location for detachably securing a needle or needle/hub assembly.

It will be appreciated that the nature of the device is limited only in the sense that drug delivery is activated by the dose button. Thus, for example, the device could be one for administering one or more medicaments, for example from a single or a plurality of replaceable cartridges mounted in the housing.

The term “medicament delivery device” as used herein, means a device capable of administering a dose of one or more medicaments to a patient. Such devices may administer fixed and/or variable doses of medicament to a patient. Handheld medicament delivery devices are sometimes called ‘pen-type’ devices. The medicament delivery mechanism employed by such devices is preferably electromechanical, utilising a motor and gearing to drive a piston rod, although manual delivery mechanisms incorporated into electrically controlled or configured devices may also be envisaged.

The term “drug” or “medicament”, as used herein, means a pharmaceutical formulation containing at least one pharmaceutically active compound,

wherein in one embodiment the pharmaceutically active compound has a molecular weight up to 1500 Da and/or is a peptide, a protein, a polysaccharide, a vaccine, a DNA, a RNA, an enzyme, an antibody or a fragment thereof, a hormone or an oligonucleotide, or a mixture of the above-mentioned pharmaceutically active compounds,

wherein in a further embodiment the pharmaceutically active compound is useful for the treatment and/or prophylaxis of diabetes mellitus or complications associated with diabetes mellitus such as diabetic retinopathy, thromboembolism disorders such as deep vein or pulmonary thromboembolism, acute coronary syndrome (ACS), angina, myocardial infarction, cancer, macular degeneration, inflammation, hay fever, atherosclerosis and/or rheumatoid arthritis,

wherein in a further embodiment the pharmaceutically active compound comprises at least one peptide for the treatment and/or prophylaxis of diabetes mellitus or complications associated with diabetes mellitus such as diabetic retinopathy,

wherein in a further embodiment the pharmaceutically active compound comprises at least one human insulin or a human insulin analogue or derivative, glucagon-like peptide (GLP-1) or an analogue or derivative thereof, or exedin-3 or exedin-4 or an analogue or derivative of exedin-3 or exedin-4.

Insulin analogues are for example Gly(A21), Arg(B31), Arg(B32) human insulin; Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Insulin derivates are for example B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-Y-glutamyl)-des(B30) human insulin; B29-N-(N-lithocholyl-Y-glutamyl)-des(B30) human insulin; B29-N-(ω-carboxyheptadecanoyl)-des(B30) human insulin and B29-N-(ω-carboxyhepta [symbol] decanoyl) human insulin.

Exendin-4 for example means Exendin-4(1-39), a peptide of the sequence H His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Pro-Ser-NH2.

Exendin-4 derivatives are for example selected from the following list of compounds:

### H-(Lys)4-des Pro36, des Pro37 Exendin-4(1-39)-NH2,

### H-(Lys)5-des Pro36, des Pro37 Exendin-4(1-39)-NH2,

### des Pro36 [Asp28] Exendin-4(1-39),

### des Pro36 [IsoAsp28] Exendin-4(1-39),

### des Pro36 [Met(O)14, Asp28] Exendin-4(1-39),

### des Pro36 [Met(O)14, IsoAsp28] Exendin-4(1-39),

### des Pro36 [Trp(O2)25, Asp28] Exendin-4(1-39),

### des Pro36 [Trp(O2)25, IsoAsp28] Exendin-4(1-39),

### des Pro36 [Met(O)14 Trp(O2)25, Asp28] Exendin-4(1-39),

### des Pro36 [Met(O)14 Trp(O2)25, IsoAsp28] Exendin-4(1-39); or

### des Pro36 [Asp28] Exendin-4(1-39),

### des Pro36 [IsoAsp28] Exendin-4(1-39),

### des Pro36 [Met(O)14, Asp28] Exendin-4(1-39),

### des Pro36 [Met(O)14, IsoAsp28] Exendin-4(1-39),

### des Pro36 [Trp(O2)25, Asp28] Exendin-4(1-39),

### des Pro36 [Trp(O2)25, IsoAsp28] Exendin-4(1-39),

### des Pro36 [Met(O)14 Trp(O2)25, Asp28] Exendin-4(1-39),

### des Pro36 [Met(O)14 Trp(O2)25, IsoAsp28] Exendin-4(1-39),

wherein the group -Lys6-NH2 may be bound to the C-terminus of the Exendin-4 derivative;
or an Exendin-4 derivative of the sequence

### H-(Lys)6-des Pro36 [Asp28] Exendin-4(1-39)-Lys6-NH2,

### des Asp28 Pro36, Pro37, Pro38Exendin-4(1-39)-NH2,

### H-(Lys)6-des Pro36, Pro38 [Asp28] Exendin-4(1-39)-NH2,

### H-Asn-(Glu)5des Pro36, Pro37, Pro38 [Asp28] Exendin-4(1-39)-NH2,

### des Pro36, Pro37, Pro38 [Asp28] Exendin-4(1-39)-(Lys)6-NH2,

### H-(Lys)6-des Pro36, Pro37, Pro38 [Asp28] Exendin-4(1-39)-(Lys)6-NH2,

### H-Asn-(Glu)5-des Pro36, Pro37, Pro38 [Asp28] Exendin-4(1-39)-(Lys)6-NH2,

### H-(Lys)6-des Pro36 [Trp(O2)25, Asp28] Exendin-4(1-39)-Lys6-NH2,

### H-des Asp28 Pro36, Pro37, Pro38 [Trp(O2)25] Exendin-4(1-39)-NH2,

### H-(Lys)6-des Pro36, Pro37, Pro38 [Trp(O2)25, Asp28] Exendin-4(1-39)-NH2,

### H-Asn-(Glu)5-des Pro36, Pro37, Pro38 [Trp(O2)25, Asp28] Exendin-4(1-39)-NH2,

### des Pro36, Pro37, Pro38 [Trp(O2)25, Asp28] Exendin-4(1-39)-(Lys)6-NH2,

### H-(Lys)6-des Pro36, Pro37, Pro38 [Trp(O2)25, Asp28] Exendin-4(1-39)-(Lys)6-NH2,

### H-Asn-(Glu)5-des Pro36, Pro37, Pro38 [Trp(O2)25, Asp28] Exendin-4(1-39)-(Lys)6-NH2,

### H-(Lys)6-des Pro36 [Met(O)14, Asp28] Exendin-4(1-39)-Lys6-NH2,

### des Met(O)14 Asp28 Pro36, Pro37, Pro38 Exendin-4(1-39)-NH2,

H-(Lys)6-desPro36, Pro37, Pro38 [Met(O)14, Asp28] Exendin-4(1-39)-NH2,

### H-Asn-(Glu)5-des Pro36, Pro37, Pro38 [Met(O)14, Asp28] Exendin-4(1-39)-NH2,

### des Pro36, Pro37, Pro38 [Met(O)14, Asp28] Exendin-4(1-39)-(Lys)6-NH2,

### H-(Lys)6-des Pro36, Pro37, Pro38 [Met(O)14, Asp28] Exendin-4(1-39)-(Lys)6-NH2,

### H-Asn-(Glu)5 des Pro36, Pro37, Pro38 [Met(O)14, Asp28] Exendin-4(1-39)-(Lys)6-NH2,

### H-Lys6-des Pro36 [Met(O)14, Trp(O2)25, Asp28] Exendin-4(1-39)-Lys6-NH2,

### H-des Asp28 Pro36, Pro37, Pro38 [Met(O)14, Trp(O2)25] Exendin-4(1-39)-NH2,

### H-(Lys)6-des Pro36, Pro37, Pro38 [Met(O)14, Asp28] Exendin-4(1-39)-NH2,

### H-Asn-(Glu)5-des Pro36, Pro37, Pro38 [Met(O)14, Trp(O2)25, Asp28] Exendin-4(1-39)-NH2,

### des Pro36, Pro37, Pro38 [Met(O)14, Trp(O2)25, Asp28] Exendin-4(1-39)-(Lys)6-NH2,

### H-(Lys)6-des Pro36, Pro37, Pro38 [Met(O)14, Trp(O2)25, Asp28] Exendin-4(S1-39)-(Lys)6-NH2,

### H-Asn-(Glu)5-des Pro36, Pro37, Pro38 [Met(O)14, Trp(O2)25, Asp28] Exendin-4(1-39)-(Lys)6-NH2;

or a pharmaceutically acceptable salt or solvate of any one of the afore-mentioned Exedin-4 derivative.

Hormones are for example hypophysis hormones or hypothalamus hormones or regulatory active peptides and their antagonists as listed in Rote Liste, ed. 2008, Chapter 50, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, Goserelin.

A polysaccharide is for example a glucosaminoglycane, a hyaluronic acid, a heparin, a low molecular weight heparin or an ultra low molecular weight heparin or a derivative thereof, or a sulphated, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium.

Antibodies are globular plasma proteins (˜150 kDa) that are also known as immunoglobulins which share a basic structure. As they have sugar chains added to amino acid residues, they are glycoproteins. The basic functional unit of each antibody is an immunoglobulin (Ig) monomer (containing only one Ig unit); secreted antibodies can also be dimeric with two Ig units as with IgA, tetrameric with four Ig units like teleost fish IgM, or pentameric with five Ig units, like mammalian IgM.

The Ig monomer is a “Y”-shaped molecule that consists of four polypeptide chains; two identical heavy chains and two identical light chains connected by disulfide bonds between cysteine residues. Each heavy chain is about 440 amino acids long; each light chain is about 220 amino acids long. Heavy and light chains each contain intrachain disulfide bonds which stabilize their folding. Each chain is composed of structural domains called Ig domains. These domains contain about 70-110 amino acids and are classified into different categories (for example, variable or V, and constant or C) according to their size and function. They have a characteristic immunoglobulin fold in which two β sheets create a “sandwich” shape, held together by interactions between conserved cysteines and other charged amino acids.

There are five types of mammalian Ig heavy chain denoted by α, δ, ε, γ, and μ. The type of heavy chain present defines the isotype of antibody; these chains are found in IgA, IgD, IgE, IgG, and IgM antibodies, respectively.

Distinct heavy chains differ in size and composition; α and γ contain approximately 450 amino acids and δ approximately 500 amino acids, while μ and ε have approximately 550 amino acids. Each heavy chain has two regions, the constant region (CH) and the variable region (VH). In one species, the constant region is essentially identical in all antibodies of the same isotype, but differs in antibodies of different isotypes. Heavy chains γ, α and δ have a constant region composed of three tandem Ig domains, and a hinge region for added flexibility; heavy chains μ and ε have a constant region composed of four immunoglobulin domains. The variable region of the heavy chain differs in antibodies produced by different B cells, but is the same for all antibodies produced by a single B cell or B cell clone. The variable region of each heavy chain is approximately 110 amino acids long and is composed of a single Ig domain.

In mammals, there are two types of immunoglobulin light chain denoted by λ and κ. A light chain has two successive domains: one constant domain (CL) and one variable domain (VL). The approximate length of a light chain is 211 to 217 amino acids. Each antibody contains two light chains that are always identical; only one type of light chain, κ or λ, is present per antibody in mammals.

Although the general structure of all antibodies is very similar, the unique property of a given antibody is determined by the variable (V) regions, as detailed above. More specifically, variable loops, three each the light (VL) and three on the heavy (VH) chain, are responsible for binding to the antigen, i.e. for its antigen specificity. These loops are referred to as the Complementarity Determining Regions (CDRs). Because CDRs from both VH and VL domains contribute to the antigen-binding site, it is the combination of the heavy and the light chains, and not either alone, that determines the final antigen specificity.

An “antibody fragment” contains at least one antigen binding fragment as defined above, and exhibits essentially the same function and specificity as the complete antibody of which the fragment is derived from. Limited proteolytic digestion with papain cleaves the Ig prototype into three fragments. Two identical amino terminal fragments, each containing one entire L chain and about half an H chain, are the antigen binding fragments (Fab). The third fragment, similar in size but containing the carboxyl terminal half of both heavy chains with their interchain disulfide bond, is the crystallizable fragment (Fc). The Fc contains carbohydrates, complement-binding, and FcR-binding sites. Limited pepsin digestion yields a single F(ab′)2 fragment containing both Fab pieces and the hinge region, including the H-H interchain disulfide bond. F(ab′)2 is divalent for antigen binding. The disulfide bond of F(ab′)2 may be cleaved in order to obtain Fab′. Moreover, the variable regions of the heavy and light chains can be fused together to form a single chain variable fragment (scFv).

Pharmaceutically acceptable salts are for example acid addition salts and basic salts. Acid addition salts are e.g. HCl or HBr salts. Basic salts are e.g. salts having a cation selected from alkali or alkaline, e.g. Na+, or K+, or Ca2+, or an ammonium ion N+(R1)(R2)(R3)(R4), wherein R1 to R4 independently of each other mean: hydrogen, an optionally substituted C1-C6-alkyl group, an optionally substituted C2-C6-alkenyl group, an optionally substituted C6-C10-aryl group, or an optionally substituted C6-C10-heteroaryl group. Further examples of pharmaceutically acceptable salts are described in “Remington's Pharmaceutical Sciences” 17. ed. Alfonso R. Gennaro (Ed.), Mark Publishing Company, Easton, Pa., U.S.A., 1985 and in Encyclopaedia of Pharmaceutical Technology.

Pharmaceutically acceptable solvates are for example hydrates.

### BRIEF DESCRIPTION OF THE FIGURES

Embodiments of then invention will now be described by way of example only with reference to the accompanying drawings in which

FIG. 1 is a plan view of a medicament delivery device according to the present invention;

FIG. 2 is a perspective view of the dose button of the device of FIG. 1;

FIG. 3 is a simplified cross-sectional view of the dose button assembly of the device of FIG. 1; and

FIG. 4 is a sectioned isometric 3D view of a detail of the device of FIG. 1, showing only the front housing part and dose button.

### DETAILED DESCRIPTION

References to the device in the following detailed description are intended to refer to the device as referenced in the appended figures and not to when the device is in a use state. Furthermore, the figures are intended to be schematic representations to highlight relevant functionality of the present invention and therefore unnecessary structures have been omitted from the device for clarity. The relative dimensions of the device are also illustratory only. Reference to ‘distal’ and ‘proximal’ are intended to refer to the end of the device where medicament delivery occurs and the opposite end pointing away from the delivery site, respectively.

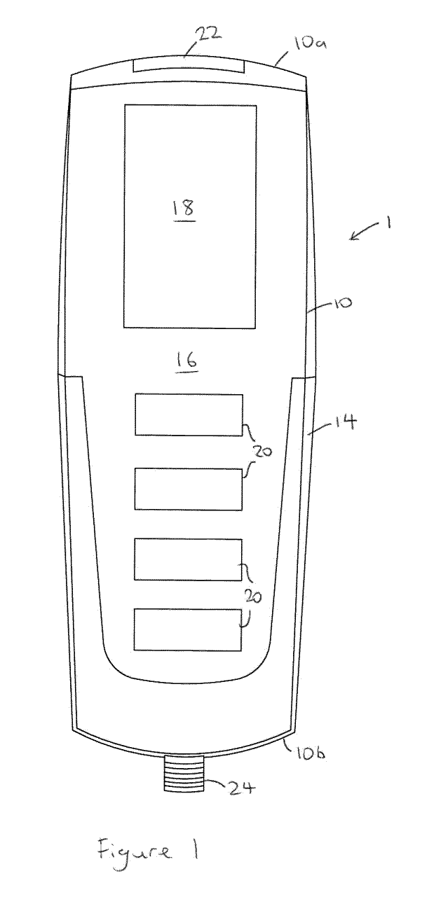

The medicament delivery device 1 illustrated in FIG. 1 comprises a housing 10 having a proximal end 10a and a distal end 10b. At the distal end 10b, the housing is shaped to receive a removable end cap or cover 12 *not shown). This end cap 12 and the housing 10 (at its proximal end) are shaped to provide a form fit connection so that once the cap 12 is slid onto the distal end 10b of the housing 10, the frictional fit between the cap 12 and the housing 10 prevents the cap from inadvertently falling off the housing 10. It will be understood that in other embodiments (not shown) other means of releasably securing the cap to the housing such as snap-fit may be employed.

The interior surface of the cap 12 and the outer surface of the housing 10 at its proximal end 10b are shaped such that there is only one possible configuration in which the cap 12 properly fits onto the distal end 10b of the housing 10. Such an arrangement is preferable because it provides certainty in the alignment of components of the cap 12 with components of the housing 10, as will be explained below.

The housing 10 contains a micro-processor control unit, a printed circuit board (PCB), an electro-mechanical drive train, a battery, and at least one medicament reservoir. A cartridge holder 14 can be removably attached to the housing 10 and may contain one or more cartridges of medicament. The cartridge holder 14 is configured so as allow the replacement of the medicament cartridges as necessary. The medicament delivery device 1 can be used to administer a computed dose of a medicament (or medicaments) through a needle assembly, such as a double ended needle assembly. It will be understood that the cap and housing arrangement described are equally applicable to needleless jet injectors.

A control panel region is provided on one major face 16 of the housing 10 and comprises a digital OLED display 18 towards the distal end 10a of the housing 10 along with a plurality of human interface elements (buttons 20 in the embodiment shown) that can be manipulated by a user to set and inject a medicament dose. It will be understood that in other embodiments (not shown) different display technology such as LCD displays can be used. The buttons 20 also allow navigation through menu structures displayed on the OLED display 18. A dose button 22 (described in more detail below) is provided in a minor face of the housing 10 at its proximal end 10a. At the distal end 10b of the housing is provided a screw-threaded needle mount 24. The needle mount 24 is configured to receive a needle hub (not shown). This needle hub can be configured to allow a dose dispenser, such as a conventional pen type injection needle assembly, to be removably mounted to the housing 10. It will be understood that the attachment between the needle mount 24 and a needle hub is preferably a screw fit to allow standard ‘type A’ needles to be fitted to the needle mount 24, although other attachment mechanisms as known in the art, such as Luer lock attachments may be used in other embodiments (not shown).

In use, when the device is turned on, the digital display 18 shown in FIG. 1 illuminates and provides the user certain device information, preferably information relating to the medicaments contained within the cartridge holder 14. For example, the user is provided with certain information relating to both the contents of the cartridge and previous dose history.

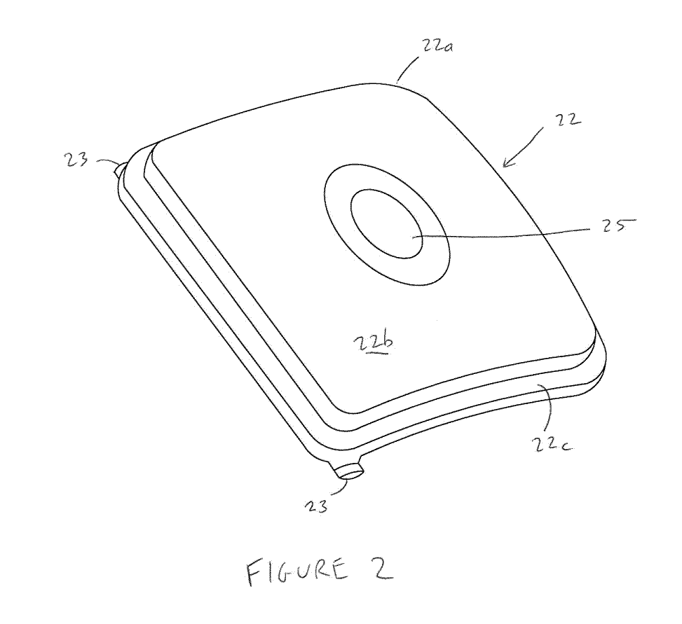

In FIG. 2, the dose button 22 is shown in perspective view. The button 22 is generally rectangular with rounded corners 22a. The button 22 has a main upper (contact) surface 22b which is downwardly stepped to form a rim 22c extending all the way around the upper surface 22b. At each side of the rim 22c towards the front of the device a round pin 23 extends outwardly. The upper surface 22b of the button 22 may be provided with a symbol 25, for example a logo consisting of two concentric ovals or circles in its centre. Such symbol 25 can be formed by for example in mould labelling. Although not shown in FIG. 2, a peg 22d extends downwardly from an undersurface of the button 22 at its centre.

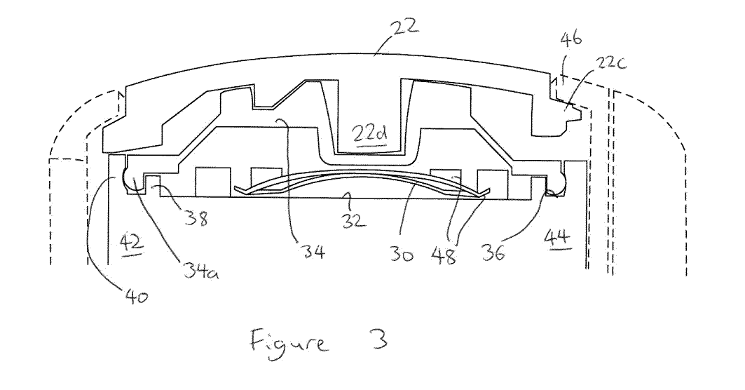

FIG. 3 is a cross-sectional view of the device along the major axis of the device 1 and through the front and rear faces of the device 1. This cross-sectional view details the structure of the dose button 22 and ancillary components. A dome switch 30 is mounted on a horizontal support surface 32 formed within the housing 10. Mounted over the dome switch 30 is a silicone gasket 34 having a downwardly depending peripheral flange 34a which is seated in a peripheral groove 36 defined by inner and outer spaced apart upstanding walls 38, 40 in front and rear housing parts 42, 44 of the horizontal support surface 32. Both the peripheral flange 34a and the peripheral groove 36 in which the flange is seated generally extend around the device 1 in the profile of the dose button 22. As well as sealing the housing 10 against the ingress of dust and moisture, the gasket 34 provides a soft tactile feel to the button 22. The button 22 itself is seated over the gasket 34 which is suitably shaped to receive the peg 22d which sits directly over the dome switch 30. The button 22 is retained in the housing 10 by a housing bezel 46, the rim 22c of the button 22 resting under the bezel 46. Also mounted on the support surface 32 are a number of LEDs 48 which provide illumination to the button 22.

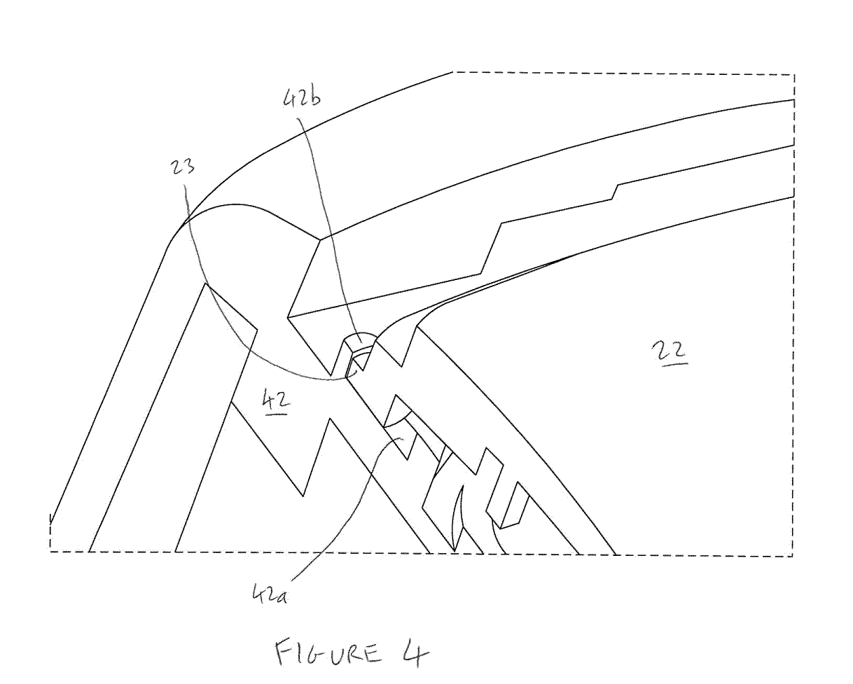

FIG. 4 is a sectioned isometric view of the button 22 mounted within the front housing part with the other components removed, the section taken parallel to the front face and towards the front of the device and illustrates how the button is hinged in use. The front housing part 42 is provided with a pair of part circular guide channels 42a, one on each side of the front housing part 42 (only one shown in FIG. 4) each of which receives a respective one of the pins 23 on the dose button 22. Each guide channel 42a has at its outer end an end stop 42b which abuts the end of the pin 23 and prevents lateral movement of the button 22. It will be understood that the guide channels 42a and pins 23 constitute hinges about which the button 22 can pivot in use.

In use, the dose button 22 is intended to be pressed by the user to commence the ejection of medicament from the medicament cartridges contained within the cartridge holder 14. As such, it is important that the dose button 22 is able to be pressed by the user from multiple directions so that the display 18 of the device 1 can preferably be viewable by the user during injection. As the user presses against the contact surface 22b, the dose button 22 is confined to pivoting about the hinges defined by the button pins 23 and guide channels 42a. This pivoting action moves the peg 22d and the interposed gasket 34 against the dome switch 30. Compression of the dome switch 30 by the action of the peg 22d actuates a mechanical switch/sensor (not shown) that sends a signal to the micro-processor control unit that the dose button 22 has been pressed. This is then used to confirm to the device that an action can be initiated (e.g. dosing).

Furthermore, allowing the dose button to be pressed from any angle allows the user to actuate the dose button 22 when the device 1 is held in any configuration. For example, a user may actuate the dose button 22 from behind using their thumb or index finger, or from the side again using their thumb or index finger. The present invention allows both configurations to actuate the button.

As the present invention limits motion of the button 22 to pivoting about a single axis, pressure on any portion of the contact surface 22b produces an identical response from the button 22. This consistent behaviour of the dose button 22 and the dome switch 30 allows only a single switch/sensor to be deployed within the dose button 22. This is in contrast to standard floating buttons in which travel of the button produces less consistent results, resulting in an increase in ‘dead’ areas (areas on the contact surface which, if pressed, produce no actuation of the underlying switch). To avoid such dead areas, either the size of the button must be reduced or the number of switches increased.

In other embodiments (not shown) the dose button is integrally formed with the housing bezel and the pins and guide channels are omitted. In such embodiments a line of weakness such as a thin walled region is provided between the housing bezel and the dose button along its front edge to serve as a hinge, the remaining edges of the button being free. One advantage of such an arrangement is that the crevice formed at the hinge is eliminated, reducing further the potential for ingress of dust and/or water.

1-11. (canceled)

12. A hand-held medicament delivery device having a housing in which is mounted,
a switch,
an electro-mechanical drug delivery mechanism activatable by the switch,
a dose button associated with the switch such that pressing of the dose button by a user activates the switch,
wherein the dose button is hinged along one edge thereof.

13. The device of claim 12, wherein the device housing has a front face incorporating a graphical display and an end face incorporating the dose button contiguous with and substantially perpendicular to the front face, the dose button being hinged substantially on the interface between the front and end faces.

14. The device of claim 1, wherein a hinge is formed by mounting the dose button on a support element of the device housing.

15. The device of any claim 12, wherein the dose button is integrally formed with at least a part of the housing, a line of weakness such as a thin walled region being provided between the housing and the dose button to serve as a hinge.

16. The device of claim 12, wherein the switch is a single dome switch.

17. The device of claim 12, wherein a gasket is provided between the switch and the dose button.

18. The device of claim 12, wherein the gasket is in the form of a flexible silicone based membrane.

19. The device of claim 12, wherein the dose button occupies at least 40% of the face of the device in which it is located.

20. The device of claim 12, wherein the device also includes a programmable microprocessor having memory and input/output functions.

21. The device of claim 12, wherein the device also includes one or more additional buttons serving as a user interface for programming the device.

22. The device of claim 12, wherein the device is a needle injection device, the housing having a mounting location for detachably securing a needle or needle/hub assembly.
